(19) United States
(12) Patent Application Publication
(10) Pub. No.: US 2019/0183394 A1
(43) Pub. Date: Jun. 20, 2019

(54) BIOSENSOR DISEASE AND INFECTION SCREENING

(71) Applicant: Eccrine Systems, Inc., Cincinnati, OH (US)

(72) Inventors: Robert Beech, Cincinnati, OH (US); Evyn White, Cincinnati, OH (US); Benjamin Katchman, Tucson, AZ (US)

(73) Assignee: Eccrine Systems, Inc., Cincinnati, OH (US)

(21) Appl. No.: 16/322,943

(22) PCT Filed: Aug. 2, 2017
(86) PCT No.: PCT/US17/45121

§ 371 Date: Feb. 2, 2019

Related U.S. Application Data
(60) Provisional application No. 62/370,232, filed on Aug. 2, 2016.

(51) Int. Cl. A61B 5/1477; G01N 33/573; G01N 33/543; A61B 5/145; A61B 5/00

(52) U.S. Cl. CPC A61B 5/1477; G01N 33/573; A61B 5/6833; A61B 5/14507; A61B 5/412; G01N 33/543

(57) ABSTRACT

The disclosed invention includes methods to employ wearable biosensing devices to accomplish the following: 1) screen for the presence of a disease or infection, including pre-symptomatic detection, and determination of the type (e.g., viral, bacterial, or fungal) of disease or infection present; 2) confirm the antigen and monitor the progress of an active infection; and 3) monitor the efficacy of a treatment program for a disease or infection. Such methods rely on the detection, in sweat or other biofluids, of proteins, nucleotides, DNA polymerases, proteases, group-specific antigens, antibodies, cytokines, or other molecules, that are produced by the body as part of the innate or adaptive immune responses to an infectious agent, or the infectious agent itself, its products, or derivatives. Some embodiments comprise a method of using such a biosensing device as a biosentinel against biological threats.

### CROSS-REFERENCE TO RELATED APPLICATIONS

This application claims benefit of PCT/US17/45121, filed Aug. 2, 2017, and U.S. Provisional Application No. 62/370,232, filed Aug. 2, 2016, the disclosures of which are hereby incorporated by reference herein in the entirety.

### BACKGROUND OF THE INVENTION

Sweat sensing technologies have enormous potential for applications ranging from athletics, to neonatology, to pharmacological monitoring, to personal digital health, to name a few applications. Sweat contains many of the same biomarkers, chemicals, or solutes that are carried in blood and can provide significant information enabling one to diagnose illness, health status, exposure to toxins, performance, and other physiological attributes even in advance of any physical sign. Furthermore, sweat itself, the action of sweating, and other parameters, attributes, solutes, or features on, near, or beneath the skin can be measured to further reveal physiological information. However, recent progress in sweat sensing technology development has been limited to high concentration analytes (μM to mM) sampled at high sweat rates (>1 nL/min/gland) found in, for example athletic applications. Progress will be much more challenging as sweat biosensing moves towards detection of large, low concentration analytes (nM to pM and lower).

In particular, many known sensor technologies for detecting larger molecules are ill-suited for use in wearable sweat sensing, which requires sensors that permit continuous or extended use on a wearer's skin. This means that sensor modalities that require complex microfluidic manipulation, the addition of reagents, or the use of limited shelf-life components, such as antibodies, will not be sufficient for sweat sensing. What is needed is a stable, reliable, reagentless sensor that is sensitive to target analytes in sweat, while providing the level of specificity necessary to produce high predictive values during the lifespan of the sensor. One solution to this problem is the use of electrochemical aptamer-based (“EAB”) sensor technology, such as is disclosed in U.S. Pat. Nos. 7,803,542 and 8,003,374, or docked aptamer EAB biosensor technology disclosed in U.S. Provisional Application No. 62/523,835, filed Jun. 23, 2017, each of which is hereby incorporated by reference herein in its entirety.

One set of applications made possible by the advances in wearable biofluid sensing technology is early detection and screening for pathogenic infections. For example, variations of influenza, caused by member of the genera Influenzavirus A, B, and C, are responsible for seasonal epidemics resulting in over 200,000 hospitalizations and 30,000 to 50,000 deaths in the United States alone, see Vemula, S., et al., “Current Approaches for Diagnosis of Influenza Virus Infections in Humans,” Viruses, 8(4) 2016, and 375,000 annual deaths worldwide. The threat posed by other diseases with even graver pandemic potential has increased alongside population growth, globally integrated economies, and ease of air travel. Outbreaks of various types threaten to overwhelm health care systems, cripple economies and cause significant loss of life. Among these, viral hemorrhagic fevers (VHFs) are of particular concern due to their irregular and sporadic emergence in populations, making the prediction of outbreaks extremely challenging. The U.S. Centers for Disease Control (CDC) classifies most VHFs as Category-A Select Agents, which represents the highest level of risk classification. Among the microbes receiving such classification are species of Flavivirus, which include Dengue virus, West Nile virus, Yellow fever virus, and Zika virus; members of the Filovirdiae family, including members of the Ebolavirus genus, and Marburgvirus genus; as well as Alphavirus, and Chikungunya virus. The World Health Organization (WHO) estimates that more than one third of the world's population risks contracting Dengue fever, with 50 to 100 million cases per year and associated cost estimates ranging from $587 million to $1.8 billion. See Suaya, et al., “Cost of Dengue Cases in Eight Countries in the Americas and Asia: A Prospective Study,” Am. J. of Tropical Med. & Hygiene, 80(5) 2009. In addition to their unpredictable outbreak tendencies, the pandemic danger of VHFs is increased by the relatively long and highly contagious incubation period between infection and the emergence of visible symptoms, e.g., Ebola (2-21 days), Dengue (3-15 days), Marburg (5-10 days), and Chikungunya (3-7 days).

State of the art field analysis techniques for early disease detection are inadequate. Current systems such as the U.S. military's Joint Biological Agent Identification and Diagnostic System (JBAIDS), rely heavily on field assay technologies, and are only capable of identifying infection after symptoms present, rather than identifying infection during the crucial pre-symptomatic stage. Further, there are no definitive tests that distinguish between viral and bacterial infections, other than testing for all possible pathogens, which is expensive, time-consuming, and impractical for screening large numbers of people. Accurate and early diagnosis of viral infections is critical for rapid initiation of antiviral therapy to reduce infection-related mortality during seasonal and sporadic epidemics and pandemics. Early screening is therefore crucial for patients with Influenza, Ebola, Zika, or others, and could help slow or thwart the development of large-scale outbreaks.

Methods for use of wearable, non-invasive biosensors as disclosed herein, present an opportunity to perform such early screening functions. As an initial matter, eccrine sweat, as an excretory biofluid, is an ideal potential source for infection byproducts exiting the body at an early stage of infection. The human body's infection response involves complex, highly regulated processes, including innate and adaptive immune responses, which result in large numbers of deployed host proteins that emerge in the bloodstream. Further, specific host proteins circulating in the bloodstream or present in the mucosa that have been induced from a viral infection are likely to be present in a sweat sample. Therefore, a method providing immediate results and utilizing a sensing device for a non-invasive biofluid, such as sweat, can provide a much-needed early warning for diagnosing or screening for infection in large numbers of people simultaneously.

Therefore, the disclosed invention includes methods capable of noninvasively monitoring individuals and generating early warning alerts that indicate the presence or type of infection within the individuals, where the potential warning period begins at infection and continues through the presentation of visible symptoms of infection. Such an invention could also perform a screening function to be used in conjunction with existing field assay systems to indicate which individuals should receive further testing.

Many of the challenges to successful biofluid sensing device development for such purposes can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sweat to sensors and sample preparing or concentrating subsystems.

### SUMMARY OF THE INVENTION

The disclosed invention includes methods to employ biosensing devices to accomplish the following: 1) screen for the presence of a disease or infection, including pre-symptomatic detection, and determination of the type (e.g., viral, bacterial, or fungal) of disease or infection present; 2) confirm the antigen and monitor the progress of an active infection; and 3) monitor the efficacy of a treatment program for a disease or infection. Such methods rely on the detection, in sweat or other biofluids, of analytes that are produced by the body as part of the innate or adaptive immune responses to an infectious agent, or the infectious agent itself, its products, or derivatives. Some embodiments comprise a method of using such a biosensing device as a biosentinel against biological threats.

### BRIEF DESCRIPTION OF THE DRAWINGS

The objects and advantages of the present invention will be further appreciated in light of the following detailed descriptions and drawings in which:

FIG. 1 depicts an example previously disclosed biofluid sensing device for use with the disclosed invention.

FIG. 2 depicts a previously-disclosed aptamer sensing element configured as part of a multiple capture EAB biosensor.

FIG. 3 depicts a previously-disclosed aptamer sensing element configured as part of a docked aptamer EAB biosensor.

FIG. 4 depicts a previously-disclosed example biofluid sensing device for use with embodiments of the disclosed invention.

FIG. 5 depicts a previously-disclosed example biofluid sensing device employing sweat sample concentration for use with embodiments of the disclosed invention.

### DEFINITIONS

Before continuing, a variety of definitions should be made, these definitions gaining further appreciation and scope in the detailed description and embodiments of the present disclosure.

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

“Biofluid” means any human biofluid, including, without limitation, sweat, interstitial fluid, blood, plasma, serum, tears, and saliva.

“Biosensor” means any type of sensor that measures a state, presence, flow rate, solute concentration, solute presence, in absolute, relative, trending, or other ways in a biofluid. Biosensors can include, for example, potentiometric, amperometric, impedance, optical, mechanical, antibody, peptide, aptamer, or other means known by those skilled in the art of sensing or biosensing.

“Analyte” means a substance, molecule, ion, or other material that is measured by a fluid sensing device.

“Measured” can imply an exact or precise quantitative measurement and can include broader meanings such as, for example, measuring a relative amount of change of something. Measured can also imply a binary or qualitative measurement, such as ‘yes’ or ‘no’ type measurements.

“Chronological assurance” means the sampling rate or sampling interval that assures measurement(s) of analytes in sample in terms of the rate at which measurements can be made of new fluid analytes as they enter the sample. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5- to 30-minute lag time between analytes in blood emerging in interstitial fluid), but the resulting sampling interval is independent of lag time, and furthermore, this lag time is inside the body, and therefore, for chronological assurance as defined above and interpreted herein, this lag time does not apply.

“EAB sensor” means an electrochemical aptamer-based biosensor that is configured with multiple aptamer sensing elements that, in the presence of a target analyte in a fluid sample, produce a signal indicating analyte capture, and which signal can be added to the signals of other such sensing elements, so that a signal threshold may be reached that indicates the presence of the target analyte. Such sensors can be in the forms disclosed in U.S. Pat. Nos. 7,803,542 and 8,003,374 (the “Multi-capture Aptamer Sensor” (MCAS)), or in U.S. Provisional Application No. 62/523,835 (the “Docked Aptamer Sensor” (DAS)).

“Biofluid sensor data” means all the information collected by biofluid sensing device sensor(s) and communicated to a user or a data aggregation location.

“Correlated aggregated biofluid sensor data” means biofluid sensor data that has been collected in a data aggregation location and correlated with outside information such as time, temperature, weather, location, user profile, other biofluid sensor data, or any other relevant data.

“Sweat generation rate” is the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

“Sweat volume” is the fluidic volume in a space that can be defined multiple ways. Sweat volume may be the volume that exists between a sensor and the point of generation of sweat or a solute moving into or out of sweat from the body or from other sources. Sweat volume can include the volume that can be occupied by sweat between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

“Microfluidic components” are channels in polymer, textiles, paper, or other components known in the art of microfluidics for guiding movement of a fluid or at least partial containment of a fluid.

“Sweat sampling rate” is the effective rate at which new sweat or sweat solutes, originating from the sweat gland or from skin or tissue, reaches a sensor which measures a property of sweat or its solutes. Sweat sampling rate, in some cases, can be far more complex than just sweat generation rate.

“Sweat stimulation” is the direct or indirect causing of sweat generation by any external stimulus, the external stimulus being applied for the purpose of stimulating sweat. One example of sweat stimulation is the administration of a sweat stimulant such as pilocarpine. Going for a jog, which stimulates sweat, is only sweat stimulation if the subject jogging is jogging for the purpose of stimulating sweat.

“Sensitivity” means the change in output of the sensor per unit change in the parameter being measured. The change may be constant over the range of the sensor (linear), or it may vary (nonlinear).

“Signal threshold” means the combined strength of signal-on indications produced by a plurality of aptamer sensing elements that indicates the presence of a target analyte.

### DETAILED DESCRIPTION OF THE INVENTION

The detailed description of the disclosed invention will be primarily, but not entirely, limited to devices and methods using wearable biofluid sensing devices. Therefore, although not described in detail here, other essential steps which are readily interpreted from or incorporated along with the present invention shall be included as part of the disclosed invention. The disclosed invention applies at least to any type of biofluid sensing device that measures a biofluid sample, a biofluid generation rate, chronological assurance, biofluid solutes, a property of or things on the surface of skin, or properties or things beneath the skin. The invention applies to devices which can take on a number of forms including patches, bands, straps, portions of clothing, wearables, or any suitable mechanism that reliably brings biofluid sensing technology into intimate proximity with the biofluid as it is generated.

Certain embodiments of the invention show sensors as simple individual components. It is understood that many sensors require two or more electrodes, reference electrodes, or additional supporting technology or features that are not captured in the description herein. Sensors are preferably electrochemical in nature, but may also include optical, chemical, mechanical, or other known biosensing mechanisms. Sensors can be in duplicate, triplicate, or more, to provide improved data and readings. Sensors may be referred to by what the sensor is sensing, for example: a sweat sensor, an impedance sensor, a sample volume sensor; or a biofluid generation rate sensor. Certain embodiments of the disclosed invention show sub-components of biofluid sensing devices requiring additional sub-components for use in various applications, which are obvious (such as a battery), and for purpose of brevity and focus on inventive aspects are not explicitly shown in the diagrams or described in the embodiments of the invention. As a further example, many embodiments of the invention could benefit from mechanical or other means known to those skilled in wearable devices, patches, bandages, and other technologies or materials affixed to skin, to keep the devices or sub-components of the skin firmly affixed to skin or with pressure favoring constant contact with skin or conformal contact with even ridges or grooves in skin, and are included within the spirit of the disclosed invention. The disclosure also includes reference to the article in press for publication in the journal IEEE Transactions on Biomedical Engineering, titled “Adhesive RFID Sensor Patch for Monitoring of Sweat Electrolytes”; the article published in the journal AIP Biomicrofluidics, 9 031301 (2015), titled “The Microfluidics of the Eccrine Sweat Gland, Including Biomarker Partitioning, Transport, and Biosensing Implications”; as well as PCT/US15/55756, each of which is included herein by reference in its entirety.

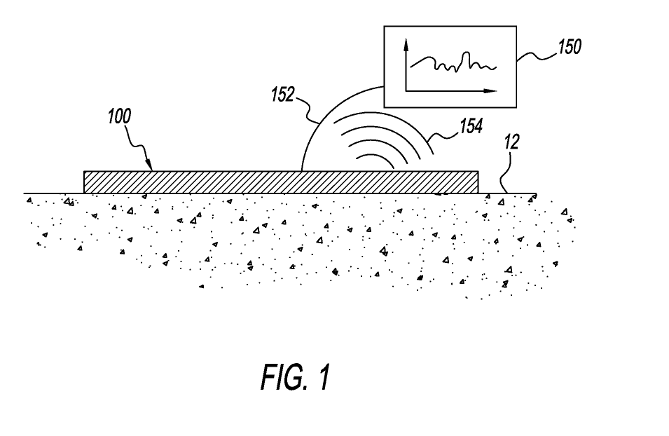

With reference to FIG. 1, a representative biofluid sensing device 100 to be used with the disclosed invention is placed on or near skin 12. The device may be fluidically connected to skin or regions near skin through microfluidics or other suitable techniques. Device 100 is in wired communication 152 or wireless communication 154 with a reader device 150, which could be a smart phone or portable electronic device, or in some embodiments the device 100 and reader device 150 can be combined. Communication 152 or 154 is not necessarily constant and could be a simple one-time data download from device 100 once it has completed its measurements of sweat or other biofluids.

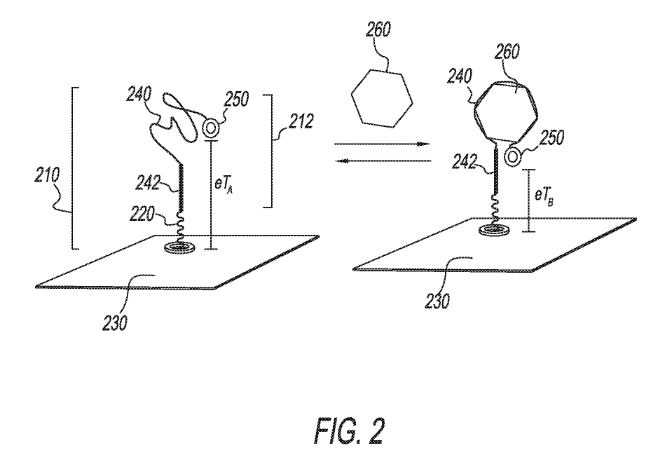

As disclosed in PCT/US17/23399, incorporated herein by reference in its entirety, some embodiments of the disclosed invention use Multi-Capture Aptamer EAB sensors to detect target analytes in biofluids. Such embodiments use EAB sensors that are configured to provide stable sensor responses with a life cycle extensive enough for multiple analyte binding and release cycles. With reference to FIG. 2, a MCAS includes a plurality of aptamer sensing elements 210 (thousands, millions, or billions of individual sensing elements, having an upper limit of 10¹⁴/cm²), each of which includes an analyte capture complex 212 that is covalently bonded to a sulfur molecule (thiol) 220, which is in turn covalently bonded to a gold electrode base. The analyte capture complex 212 is functionalized to undergo a conformational change upon analyte capture that produces a significant change in electrical signal. The complex 212 includes a selected aptamer sequence 240, one or more primer section(s) 242, and a redox moiety 250 that is bonded or linked directly to the aptamer sequence. In its free state, the aptamer sequence 240 is able to fold into its preferred configuration, and the redox moiety 250 assumes a first position relative to the electrode 230. The electrode 230 may be comprised of gold or another suitable conductive material. In this first position, the distance between the redox moiety 250 and electrode 230 is sufficiently large to prevent most electron transduction. Therefore, when the sensor interrogates the sensing element using, e.g., square wave voltammetry, the sensing element produces a first electrical signal eT_{A}. When the aptamer 240 captures a target analyte molecule 260, the aptamer undergoes a conformational change that partially disrupts the first configuration, and forms a second configuration. The capture of the target analyte 260 accordingly moves the redox moiety 250 into a second position relative to the electrode 230. Now when the sensor interrogates the sensing element, it produces a second electrical signal eT_{B} that is distinguishable from the first electrical signal eT_{A}. After a recovery interval, the complex 212 releases the target analyte, and the aptamer will return to the first configuration, which will produce the corresponding first electrical signal when the sensing element is interrogated

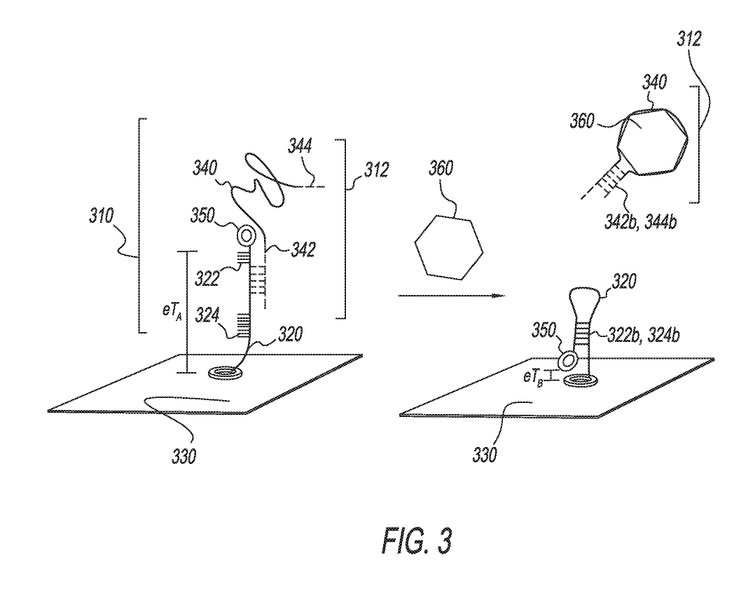

Another EAB sensor modality used with some embodiments of the disclosed invention is a Docked Aptamer EAB sensor. With reference to FIG. 3, a DAS aptamer sensing element is depicted. The aptamer sensing element 310 includes an analyte capture complex 312 and a molecular docking structure 320 immobilized on an electrode 330. While the figure depicts, and the discussion focuses on, a single aptamer sensing element, DAS described herein will include a large number (thousands, millions or billions) of such aptamer sensing elements attached to the electrode. The docking structure 320 may be attached to the electrode 330 by covalently bonding a first end to a thiol, which is then in turn covalently bonded to the electrode. The electrode 330 may be comprised of gold or another suitable conductive material. The docking structure 320 includes a 9 to 12 base nucleotide sequence that is selected to be complementary with a nucleotide sequence on the analyte capture complex 312, specifically, the dock is configured to pair with a first primer section 342. A redox chemical moiety 350 is immobilized on the unattached end of the dock 320, on the opposite end of the dock from the electrode 330. The dock 320 further includes two complementary nucleotide sequences 322, 324. In the initial arrangement, the analyte capture complex 312 is attached to a dock 320 that is, in turn, attached to the electrode 330. When the dock is bound to the analyte capture complex, it is stiffened so that the redox moiety 350 is located at a maximum distance from the electrode 330, being approximately the full length of the dock. The distance between the redox moiety and electrode is sufficiently large to prevent most electron transduction, thereby largely preventing redox of the redox moiety in response to potentials applied via electrode 330, effectively creating a no- or reduced-signal condition prior to analyte capture eT_{A}.

In operation, the DAS is exposed to a biofluid sample containing a concentration of the target analyte 360. Upon interaction with the target analyte, the aptamer 340 is drawn physically around the analyte 360 to capture the analyte, causing the second primer 344B to move into physical proximity to the first primer 342B. The physical proximity of the complementary primers causes the first primer to break free from the dock 320 and bind to the second primer 344B, and the complex is carried away from the docking structure 320. Once the dock 320 is unbound from the first primer 342B, the dock becomes more flexible, and the complementary sections 322B, 324B bind together. The folding of dock 320 caused by the sections binding locks the attached redox moiety 350 in a position close to the electrode 330, thereby promoting a detectable electron transduction eT_{B}. Interrogation of the electrode 330 following analyte capture, therefore, will return a detectable signal due to the proximity of the redox moiety to the electrode.

Detection of the target analyte will be positively indicated when a sufficient number of EAB sensing elements captures a target analyte molecule, and produces a capture signal when interrogated by the device. The strength of the signal required to indicate the presence of the target in the sweat sample is the signal threshold, which will be set by application to achieve a desired predictive value that balances false positive and false negative indications. Some applications, such as screening the general population for a heart condition, may require very low false positive indications, and therefore would need to have a higher signal threshold, representing greater certainty of analyte presence. Other applications, such as preliminary screening for Ebola exposure in an at-risk population, may not require such high certainty, and could use a lower signal threshold. In other cases, for example, an EAB sensing element may have an aptamer that relatively weakly binds the target analyte, or the particular sweat sample may have challenging pH or salinity characteristics, or the target analyte may be very dilute or very small. In each of these cases, the signal threshold would need to be relatively higher than in the converse case, all other factors being equal.

A primary difference between the operation of an MCAS as described in relation to FIG. 2, and the DAS described in relation to FIG. 3, is that the DAS sensing element releases the analyte capture complex upon interaction with a target analyte, and thus is unable to recapture a target analyte at a later time. While this characteristic limits the DAS ability to provide continuous or extended sensing of analyte concentration changes, it also presents an advantage over MCAS modalities at low analyte concentrations. At low concentrations, the MCAS sensing elements will readily release target analytes back into the low concentration solution, and return to a signal-off state. Such behavior will make it less likely that the MCAS is able to reach a signal threshold and clearly indicate the presence of a target analyte, even when the analyte is actually present in the biofluid sample. By contrast, a DAS will cumulatively register the presence of the target analyte over time, allowing the detection of low concentration analytes, as is more fully disclosed in U.S. Provisional Application No. 62/371,902, filed Aug. 8, 2016, which is hereby incorporated by reference herein in its entirety.

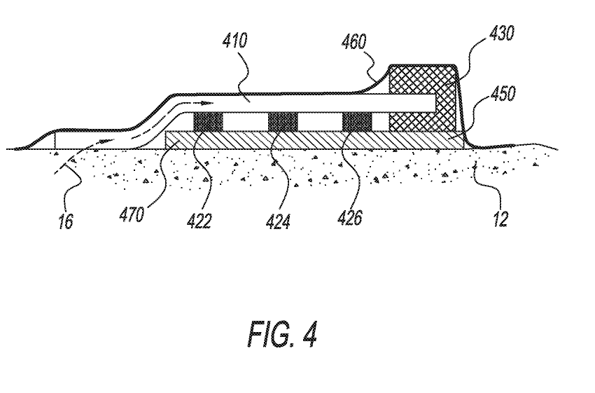

FIG. 4 is an example EAB biofluid sensing device for use with the disclosed invention. The device includes a microfluidic wicking component 410 and microfluidic pump 430 that receives sweat 16 from the wick 410. Pump 430 could be a wicking hydrogel but could also be other suitable materials. The device includes at least one EAB sensor 422, 424, and an optional secondary sensor 426 situated on a fluid impermeable substrate 450. In some embodiments, the device includes an optional protective vapor barrier layer 460 over wicking component 410, which prevents or reduces biofluid sample evaporation out of the device. In other embodiments, a vapor barrier layer (not shown) could be located above the substrate 450 and below sensors 422, 424, 426 to prevent vapor from escaping once it has entered the sweat managem cut system.

Biofluid sample concentration ranges for target analytes relevant to infection screening and detection are likely to be very low. Typical sweat concentration ranges for different analytes are in the mM range for small molecules such as cortisol, but decline to μM ranges for larger solutes, like luteinizing hormone, and can be as low as to pM and even fM ranges for larger proteins. The requirement to detect such minute levels of infection-related analytes will profoundly affect EAB sensing strategies for disease detection. When target analyte concentrations are lower, EABs will naturally have fewer capture opportunities, requiring greater sensitivity to ensure that the reduced capture opportunities are fully exploited. Aptamer selection will accordingly be required to aim for greater sensitivity for such low concentration analytes. However, despite best efforts at aptamer selection, many target analytes in sweat and other biofluids will remain below the limits of detection for EAB sensing technology. Therefore, devices for use with the disclosed invention may be configured to concentrate the biofluid samples to bring target analytes within EAB sensor detection limits, as more fully discussed in PCT/US16/58356, filed Oct. 23, 2016, which is hereby incorporated by reference herein in its entirety.

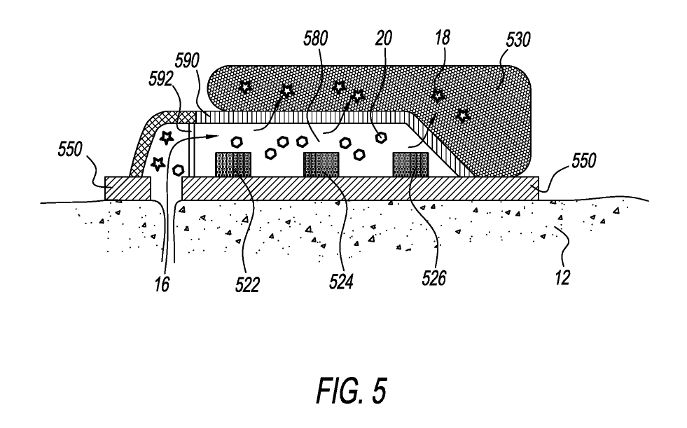

Therefore, with reference to FIG. 5, an example EAB biofluid sensing device for use with some embodiments of the disclosed invention is depicted that includes at least one EAB sensor and facilitates biofluid sample concentration. The device includes a sweat-impermeable substrate 550, that carries a concentrator channel 580, at least one EAB sensor 522, 524, a concentrator membrane 590, a pump 530, an optional secondary sensor 526, an optional pre-sensor membrane 592, and an optional protective vapor barrier layer (not shown). The device is placed on skin 12 via an adhesive (not shown). As the biofluid sample 16 enters the device and flows into the concentrator channel 580, water, and in some cases untargeted solutes 18, are drawn through the membrane 590, and into the pump 530. Such sample manipulation leaves the target analyte molecules 20 in the concentrator channel 580, and effectively concentrates the sweat sample with respect to the target analyte.

The concentrator membrane 590 is a dialysis membrane, or is an osmosis membrane permeable to ions and impermeable to small molecules and proteins, or may be a membrane that is at least permeable to water and impermeable to the target analyte. The material in the pump 530 may facilitate wicking or osmotic flow, and is a hydrogel, textile, salt, polyelectrolyte solution, or desiccant, such as MgSO₄. In some embodiments, the pump 530 would have a significantly greater volume than the concentrator channel 580 to facilitate pH and salinity buffering of the biofluid sample. The optional pre-sensor membrane 592, also made from similar material types as used for the concentrator membrane, filters unwanted solutes, such as molecules larger than the target analyte, from the biofluid sample before it reaches the EAB sensor(s) 522, 524.

Accordingly, the disclosed invention includes methods to employ the EAB biofluid sensing devices described above to 1) screen for the presence of a disease or infection, including pre-symptomatic detection, and determination of the type (e.g., viral, bacterial, or fungal) of disease or infection present; 2) confirm the antigen and monitor the progress of an active infection; and 3) monitor the efficacy of a treatment program for a disease or infection. Such methods rely on the detection, in sweat or other biofluids, of proteins, nucleotides, DNA polymerases, proteases, group-specific antigens, antibodies, cytokines, or other molecules, which are either produced by the body in response to an infectious agent, or produced by, or derived from, the infectious agent itself.

Among the human body products that may be used in wearable biofluid sensing devices for disease detection are products of the innate immune system and the adaptive immune system. The innate immune system is the first line of defense against pathogenic infections, including without limitation, viral, bacterial, and fungal infections, and is generally described as non-specific to the particular antigen invading the body, but may show variations depending on the type of infection. Innate immune response includes cell-mediated immunity, which initiates a cascade of responses including the activation of white blood cells, e.g., dendritic cells, neutrophils, mast cells, and natural killer cells, that recognize and destroy infectious material. Cell-mediated immunity also involves the activation or release of various cytokines and chemokines, which contribute to inflammation, which in turn activates other innate immune responses, such as the deployment of C-reactive protein. Cytokines also initiate other induced but non-adaptive immune responses, e.g., interferons cause the production or deployment of further products, such as select white blood cell types, and Interferon gamma-induced protein-10 (IP-10).

Many of these initial responses to infection emerge in sweat and other biofluids within the first day of infection. For example, prior to viral replication (viremia) in an infected individual, bloodstream levels of circulating immune cells increase more than 1000× compared to a healthy individual. Further, over 80 known cytokines are activated in response to viral infections, many of which are upregulated within 24 hours of an infection. Certain of these, including TNF-α, IL-1, IL-6, IL-12, IL-15, IL-18, IFN-α, IFN-β, and IFN-γ, are present at low concentrations in the plasma and sweat of healthy individuals, and thus can aid in comparison between healthy and infected sweat. See, e.g., Marques-Deak, A., et al., “Measurement of cytokines in sweat patches and plasma in healthy women: validation in a controlled study,” J. Immunol. Methods, 315(1-2):99-109, 2006. Another cytokine, TNF-related apoptosis-inducing ligand (TRAIL), normally circulates in the bloodstream as a product of healthy cells, and is upregulated in response to viral infection. TRAIL thus may be upregulated in sweat during the early stages of an infection.

Accordingly, an embodiment of the disclosed invention includes a method for performing an early screen for innate immune response that indicates the presence of an infection in a biofluid sensing device wearer, and transmits an alert message to a device user which identifies the wearer for further testing. For example, biofluid sensing devices configured with EAB sensors for IL-6, TRAIL and TNF-α could be distributed to a group of individuals just returning to the U.S. from a county suffering an Ebola outbreak. Devices would be used to monitor the individuals continuously for at least three days, and up to 14 days. The EAB sensors would measure the concentration of the three chosen biomarkers, which would be compared to a healthy baseline. The baseline value can be developed by use of the individual's healthy baseline sweat concentrations of the biomarkers, or by use of a database containing biomarker concentrations in multiple healthy individuals, for example a group of individuals located in similar geographic region, an age group, or other suitable category. If an individual's measured biomarker levels indicated the presence of an infection, that individual is then referred for further testing to determine whether the infection is Ebola. For some embodiments, healthy concentrations of the measured biomarkers may be below the EAB detection threshold, and therefore the mere detection of a biomarker in sweat represents a significant upregulation indicating the presence of an infection.

In addition to screening for the presence of an infection, the disclosed method can also be used to determine the type of infection, i.e., viral, bacterial, parasitic, or fungal. Viral infections cause the innate immune system to produce a number of products that during other types of infections either are not produced, or are produced in lower amounts. As discussed above, over 80 cytokines and certain other products, like TRAIL, are upregulated in response to viral infections. Further, there is evidence that viral infections can elicit signatures that correlate to specific antigens that are evident in the circulating immune response. For example, researchers have been able to distinguish between Lassa and Marburg viral infections based on ratios of blood concentrations of microRNAs, heat shock proteins, antibodies, and cell adhesion molecules. Detection of such viral infection signatures can reveal the presence of a viral infection within three days post-infection, and prior to viremia. See Caballero, I., et al., “Lassa and Marburg viruses elicit specific host transcriptional responses early after infection,” BMC Genomics, 2014 15:960. These signatures can also show sustained expression during the course of the disease. Several other viral infections, e.g., Ebola, influenza, HIV, and respiratory syncytial virus, display early, robust, and systemic expression of cytokines, chemokines, and white blood cells prior to the onset of clinical symptoms and viremia. See Oved, K., et al., “A Novel Host-Proteome Signature for Distinguishing between Acute Bacterial and Viral Infections,” PLOS ONE, 2015 10(3): e0120012.

Like viruses, invasive bacteria trigger innate immune responses that can carry distinctive signatures associated with the bacterial type of infection. For example, procalcitonin (PCT) is a peptide precursor of calcitonin that is upregulated in blood due to inflammation caused by bacterial infection. Healthy bloodstream concentrations of PCT are typically lower than 0.01 μg/L, but in the event of a severe bacterial infection, can increase to 100 μg/L. By contrast, PCT is not significantly upregulated in response to inflammation caused by viral infection or other causes. Meisner, M., et al., “Comparison of Procalcitonin (PCT) and C-reactive Protein (CRP) Plasma Concentrations at Different SOFA Scores During the Course of Sepsis and MODS,” Critical Care, 1999, 3(1): 45-50.

An embodiment of the disclosed invention therefore includes a method for performing an early screen for innate immune response that indicates the presence and type of infection in a biofluid sensing device wearer, and transmits an alert message to a device user. For example, biofluid sensing devices configured with EAB sensors for measuring biomarkers that show differentiation based on infection type are distributed to a group of individuals. These biomarkers include IL-6, which is generally upregulated whenever inflammation is present; TRAIL, which is upregulated in viral infections; C-reactive protein, which is upregulated in bacterial infections, and IP-10, which is upregulated in both viral and bacterial infections. Devices would be used to monitor the individuals continuously for 3 to 14 days, and compare biomarker concentrations to healthy baseline levels. By using this or a similar combination of biomarkers, the disclosed method is able to distinguish between an infected state and a non-infected state, e.g., upregulated IL-6 and IP-10, and between a bacterial and a viral infection, e.g., upregulated TRAIL and baseline C-reactive protein would indicate a viral infection. Similar groups of biomarkers can also be selected that enable distinction among parasitic and fungal infections in addition to viral and bacterial infections.

Using this or similar biomarker combinations provides other benefits as well. For example, concentrations of circulating host-proteins, such as the innate immune system products discussed above, are sensitive to interpatient variability. The use of a combination of biomarkers, each of which participates in a different immune response pathway, or results from different antigen impetus, can compensate for interpatient variability and thereby improve diagnostic accuracy. Such a diverse group of biomarkers, including unrelated biomarkers that the body expresses through different pathways and in response to different stimuli, therefore represents a preferred embodiment.

Methods of the disclosed invention may also use products of the human body's adaptive immune system, which involves the rapid proliferation of memory cells and antibodies that are specific to a particular antigen. Adaptive immune response accelerates the body's response to a recurring infection, and generates new antibodies and T-cells responsive to antigens to which the body is naive. For example, bloodstream antibody concentrations can see a 4× increase over baseline levels during convalescence from an infection. Currently, antibody detection in blood or other biofluid is not widely used for early diagnosis of infections because such tests lack the ability to analyze trends, and thus cannot distinguish among antibodies that are present due to a past infection, a past immunization, or an existing infection. Rapid antibody assay technology used for such detection only allows a qualitative (yes/no) measurement, while trend identification requires an accurate quantitative antibody assay. However, methods of the disclosed invention, which include periodic or continuous sweat monitoring and the identification of specific antigens, offer the opportunity to recognize trends in the sweat concentration of antibodies, allowing the identification of an existing infection. The full repertoire of antibody isotypes have been identified in the sweat of both healthy and sick individuals. See Metze, D., et al., “Immunohistochemical demonstration of immunoglobulin A in human sebaceous and sweat glands,” J. Invest. Dermatol., 1989 January; 92(1):13-7; Forstrom, L., et al., “IgE in human eccrine sweat,” J. Invest. Dermatol., 1975 March; 64(3):156-7. Further, specific IgG and IgA antibodies to Hepatitis B, H1N1 and H2N3 Influenza, and Epstein Barr virus have been isolated in sweat, and quantitative measurements of a broad range of antibody isotypes have been established in the sweat of in healthy individuals.

Therefore, an embodiment includes a method to use a sweat sensing device to monitor an individual, either continuously or periodically, to identify an increase in sweat antibody concentrations by comparing device measurements with a baseline value. Embodiments of the disclosed method can thus perform early screening for particular diseases, or can confirm and monitor the progress of infections through continuous or periodic monitoring of individuals. Some embodiments combine detection of disease-specific antibodies with innate immune response biomarkers to monitor an infection.

Other embodiments of the disclosed invention measure antigens, antigen components, or antigen products in sweat or other biofluids to perform disease detection. For example, viral infections may be indicated by the disclosed invention through the detection of viral products (i.e., RNA, DNA, viral proteins, glycoproteins, capsid proteins, capsomeres, lipid envelopes, viral buds, or other products) that are present in sweat. Further, viral infections may produce different products depending on the maturity or duration of the infection, which could allow the measurement of the severity or stage of an infection. Many viral infections result in detectable levels of viral products in human sweat. For instance, hepatitis C virus (HCV) infections have been shown to produce detectible levels of HCV core protein and encapsidated HCV RNA. See Ortiz-Movilla, N., et al., “Hepatitis C virus replicates in sweat glands and is released into sweat in patients with chronic hepatitis C,” J. Med. Virol. 68(4):529-36, 2002. Similarly, soluble viral antigens to hepatitis B (HBV) also emerge in sweat. See Broderson, M., et al., “Detection of HBsAg and HBsAb in sweat,” Acta. Hepatogastroenterol. (Stuttg.), 1976 June; 23(3):194-201. As has sweat-borne HBV DNA polymerase. Research also indicates that Ebolavirus components, RNA, and perhaps entire viruses are present in sweat. See Kreuels, B., et al., “A Case of Severe Ebola Virus Infection Complicated by Gram-Negative Septicemia,” N Engl. J. Med., 2014; 371:2394-2401.

Other studies indicate that Filovirus was present in sweat, although there was insufficient data to determine if the virus was Ebola or Marburg due to similarities among members of the Filovirus family. There is also indication that infections by species of Flavivirus, i.e., Zika, Dengue, result in sweat concentrations of virus, viral products, or components, such as Dengue virus-specific envelope protein (ENV), and the flavivirus NS1 protein. Similarly to distinguishing among Filoviridae members, differentiation among members of the Flaviviridae genus is challenging due to their homologous structure. The following table indicates viral components that can be used with embodiments of the disclosed method.

| Virus | Viral Component |
|---|---|
| Influenza A and B | NP, HA |
| HBV | HBcAg (capsid), HBsAg (ENV), HBeAg (core) |
| HCV | NS3, NS4, NS5, core proteins |
| HIV-1 and HIV-2 | ENV, p24, gp24 (HIV-1), gp36 (HIV-2) |
| Dengue | NS1, ENV |
| Zika | NS1, ENV |
| Ebola | glycoprotein |
| Chikungunya | NS1, ES2, ENV |

There is thus evidence suggesting that sweat may prove a valuable medium for diagnosis of viral infections. While differentiation among members within a classification of viruses, e.g., distinguishing between Influenza A and Influenza B, or between Zika and Dengue, may prove difficult for a rapid point of care diagnostic method, distinguishing among the classifications, e.g., distinguishing between a Flaviviridae infection and a Filoviridae infection, is feasible and may prove highly valuable for point of care screening. Additionally, the disclosed method can augment sweat sensing device results with outside information, such as the geographic region in which the device wearer is from or is located, the seasonal variation in viral outbreaks, a particular occurrence of an outbreak, or other suitable factor to improve accuracy. In particular, in the case of highly dangerous viruses, such as Zika or Ebola, the ability to perform rapid field diagnosis on large numbers of people remains an urgent unmet medical need.

An embodiment therefore includes a method to use a biofluid sensing device configured with EAB sensors for measuring one or more sweat products of a virus. Depending on the application, the device may detect analyte concentrations indicating exposure to a virus, an infection by a specific virus, the maturity or severity of a viral infection, or responsiveness to antiviral treatment. For example, biofluid sensing devices configured with EAB sensors for measuring flavivirus NS1, Dengue ENV, Ebola glycoprotein, and Filoviridae NS1 are distributed to a group of individuals. Devices would be used to monitor the individuals continuously for 3 to 14 days, and compare biomarker concentrations to healthy baseline levels or previous levels to identify a trend. By using this or a similar combination of biomarkers, the disclosed method is able to distinguish between a Flaviviridae infection and a Filoviridae infection, e.g., upregulated flavivirus NS1 or Dengue ENV indicates a Flaviviridae infection and upregulated Ebola glycoprotein or Filoviridae NS1, indicates a Filoviridae infection. Some embodiments combine detection of viral components with innate immune response biomarkers and/or adaptive immune response biomarkers to monitor an infection. Further, using a combination of biofluid concentrations of innate immune, adaptive immune and antigen biomarkers, some embodiments can develop and use a disease signature that indicates the presence or status of a particular infection.

In some embodiments of the disclosed invention, bacterial infections may be indicated by the disclosed invention through the detection of bacteria, bacteria components, or bacterial products that emerge in sweat in the event of an infection. For example, several types of bacteria themfolliculitis (Pseudomonas), impetigo (Staphylococcus aureus), methicillin-resistant Staphylococcus aureus and Streptococcus themselves known to emerge in sweat during an infection. Bacterial products, such as endotoxins and exotoxins, can also be used to indicate a bacterial infection. For example, Lipopolysaccharides (LPS) are bacterial toxins produced in bacterial cells, and secreted into the outside environment through vesicles, or released upon the destruction of the bacteria. LPS is upregulated in the bloodstream during a bacterial infection, and can be used to indicate the presence of a bacterial infection.

In some embodiments of the disclosed invention, fungal infections may be indicated by the disclosed invention through the detection of the fungus, fungal components, or fungal products, that emerge in sweat during an infection. For example, members of the Candida genus, such as Candida albicans, and products of eumycetoma infections are known to emerge in sweat.

Embodiments of the disclosed methods may benefit from the use of data external to a biofluid sensing device that is aggregated and correlated to a device user or application. For example, information about the device wearer or the device wearer's location can prove relevant to detecting or assessing an infection probability for an individual. An individual's age, health, nutrition level, occupation, social activities, or other factors may render that person more or less vulnerable to becoming infected by a particular antigen. Similarly, geographic factors, such as an individual's physical location, recent areas visited, or place of origin may prove relevant, as may the timing and location of disease outbreaks, and the presence or movement of disease vectors, e.g., mosquitos, ticks, rodents. The time of year can be relevant if a disease, such as influenza, displays periodic outbreak patterns. Such factors may be considered when establishing a baseline analyte concentration values for healthy individuals, when establishing a disease or infection signature, or when determining whether to send a device alert message. For example, devices used on individuals that have recently visited an area with an Ebola outbreak may have their EAB sensors set to a lower signal threshold to trigger an alert message. In such a case, the device user would be willing to accept a greater number of false positive indications because the tested population is at a higher risk level for infection.

The disclosed invention includes methods of using biofluid sensing devices that are configured to perform disease detection as described herein to facilitate biosentinel and biodefense applications. For example, individuals in southern Florida are outfitted with sweat sensing devices configured to detect Zika. Members of that group are exposed to Zika via mosquitoes and subsequently several devices send alert messages indicating the detection of a Zika infection. The alerts are received by the device wearers and the Centers for Disease Control. In addition to receiving personal early warning of infection, the wearers function as biosentinels since the CDC is also able to monitor progression of Zika and recommend countermeasures. In another example, Transportation Security Administration airport screening personnel are provided with sweat sensing devices configured to detect Bacillus anthracis immunoglobulin, LPS, C-reactive protein, and IP-10 for biodefense against a weaponized Anthrax threat. Positive indication of a bacterial infection generally or Anthrax specifically would trigger alert messages to the device wearers and the Department of Homeland Security. Wearers with positive indications are then screened further and other precautions taken.

This has been a description of the present invention along with a preferred method of practicing the present invention, however the invention itself should only be defined by the appended claims.

What is claimed is:

1. A method to screen for an infection by an antigen, comprising:
using a biosensing device that is configured to be worn on an individual's skin to take one or more measurements of an analyte in a biofluid sample, wherein the analyte is one of the following: a solute indicating the individual's innate immune response to the antigen; a solute indicating the individual's adaptive immune response to the antigen; a solute produced by the antigen; and at least a portion of the antigen;
developing a baseline value for the one or more analyte measurements that correlates to a healthy condition for the individual;
comparing the one or more analyte measurements to the baseline value to determine if the infection is indicated; and
transmitting an alert message to a device user communicating a status of the infection.

2. The method of claim 1, wherein the status of the infection is determined using one or more of the following information categories: a value that correlates to a healthy concentration of the analyte in the biofluid of the individual; a value that correlates to a healthy concentration of the analyte in the biofluid of a plurality of healthy individuals; a set of relevant physical characteristics of the individual; a geographic location of the individual; a previous geographic location of the individual; an exposure of the antigen to the individual; an outbreak of a disease caused by the antigen; one or more characteristics of the antigen; a presence of vectors that carry the antigen; and a time of year.

3. The method of claim 1, further comprising determining a type of the infection, wherein the type is one of the following: a viral infection; and a bacterial infection.

4. The method of claim 3, further comprising: taking one or more measurements of the following analytes in the biofluid sample: IL-6, TNF-related apoptosis-inducing ligand (TRAIL), C-reactive protein, and IP-10.

5. The method of claim 3, further comprising determining whether an infection is a viral infection by measuring biofluid concentrations of the following analytes: IL-6, TNF-related apoptosis-inducing ligand (TRAIL), and TNF-α.

6. The method of claim 3, further comprising determining whether an infection is a viral infection by measuring biofluid concentrations of one or more of the following analytes: TNF-α; IL-1; IL-6; IL-12; IL-15; IL-18; IFN-α; IFN-β; IFN-γ; TNF-related apoptosis-inducing ligand (TRAIL); a microRNA; a cell adhesion molecule; a circulating host protein; an antibody; a T-cell; a heat shock protein; an RNA molecule; a DNA molecule; a virus; a viral protein; a glycoprotein; a capsid protein; a capsomere; a lipid envelope; and a viral bud.

7. The method of claim 3, further comprising determining whether an infection is a bacterial infection by measuring biofluid concentrations of the following analytes: IL-6, procalcitonin (PCT), and C-reactive protein.

8. The method of claim 3, further comprising determining whether an infection is a bacterial infection by measuring biofluid concentrations of one or more of the following analytes: IP-10; IL-6; procalcitonin (PCT); C-reactive protein; a microRNA; a cell adhesion molecule; a circulating host protein; an antibody; a T-cell; a heat shock protein; RNA; DNA; a bacterium, a bacterial component; a bacterial product; a bacterial endotoxin; a bacterial exotoxin; and a lipopolysaccharide (LPS).

9. The method of claim 1, further comprising developing a disease signature that correlates to an infection by the antigen.

10. The method of claim 1, further comprising determining if an infection is indicated within one of the following time periods after the individual is exposed to the antigen: less than 24 hours; less than 48 hours; less than 72 hours; less than one week; and less than two weeks.

11. The method of claim 3, wherein the type further includes the following: a parasitic infection; and a fungal infection.

12. The method of claim 11, further comprising determining whether an infection is a fungal infection by measuring biofluid concentrations of one or more of the following analytes: a microRNA; a cell adhesion molecule; a circulating host protein; an antibody; a T-cell; a heat shock protein; RNA; DNA; a fungus, a fungal component; and a fungal product.

13. The method of claim 1, wherein the device is used to facilitate biosentinel applications, further comprising:
identifying the antigen as a biological threat;
configuring the device to screen for the antigen;
deploying the device to a plurality of individuals at risk for exposure to the antigen;
relaying the alert message to a device user tasked to respond to the biological threat.

14. A method for using a biosensing device configured to be worn on an individual's skin to monitor an infection by a specific antigen, comprising:
taking at least one measurement of the individual's biofluid for at least one analyte, where the analyte is one or more of the following: a molecule indicating the individual's innate immune response to the antigen; a molecule indicating the individual's adaptive immune response to the antigen; a molecule produced by the antigen; and at least a portion of the antigen;
developing a comparison value for the analyte measurement that correlates to a previous physical condition for the individual;
comparing the analyte measurement to the baseline to determine the infection status; and
transmitting an alert message to a device user relaying the infection status.

15. The method of claim 14, wherein the infection status includes at least one of the following: an infection maturity; an infection severity; and an effectiveness of an infection treatment.
